(19) United States
(12) Patent Application Publication
(10) Pub. No.: US 2016/0284241 A1
(43) Pub. Date: Sep. 29, 2016

(54) Methods and Systems for Simulating an X-Ray Dental Image

(71) Applicant: UNIVERSITY OF WASHINGTON THROUGH ITS CENTER FOR COMMERCIALIZATION, Seattle, WA (US)

(72) Inventor: Cleber P. SILVA, Seattle, WA (US)

(21) Appl. No.: 14/914,935

(22) PCT Filed: Aug. 29, 2014
(86) PCT No.: PCT/US14/53361

§ 371 Date: Feb. 26, 2016

Related U.S. Application Data
(60) Provisional application No. 61/871,747, filed on Aug. 29, 2013.

(51) Int. Cl. G09B 23/28; A61B 6/14

(52) U.S. Cl. CPC G09B 23/286; G09B 23/283; A61B 6/14

(57) ABSTRACT

A system and method for simulating an x-ray dental image is provided. The system comprises a simulated instructonal device including a mouth, and simulated first and second alignment devices to align a film within the mouth of the instructional device. The method comprises detecting, via a sensor associated with the mouth, a signal emitted by the first alignment device that is pointed at a location on or in the mouth. The signal indicates the location. The method may further comprise determining a position and an angulation for the detected signal, correlating the position and the angulation to an image, and displaying the image on a template.

### CROSS-REFERENCE TO RELATED APPLICATIONS

This application claims priority to U.S. Provisional Patent Application Ser. No. 61/871,747 filed on Aug. 29, 2013, which is hereby incorporated by reference in its entirety.

### BACKGROUND

Radiographic technique training is offered, and often required, for those providing dental treatment to patients.

Currently, a common training technique is to repeatedly expose a simulated patient (e.g., a dummy) to radiation until an adequate image is captured. Proper alignment of the x-ray cone with the x-ray film to achieve a correct position and angle within the simulated patient mouth to image the mouth are taught via these repeated exposures. Radiographic training may help students perform a diagnosis, treatment planning, and other evaluation of medical conditions.

The simulated patients typically include a real human maxilla and mandible to offer imaging comparable with imaging obtained from live patients. Such simulated patient mouths are cumbersome to open and place a film therein, however, due to a strong spring used to open and close the mouth. Such simulated patients with real human maxilla and mandibles are also expensive. Additionally, the training technique requires use of real dental radiology equipment often located within a real dental radiology facility, involving closure of the facility to perform the training Trainees are furthermore subjected to a repeated risk of exposure to radiation during training

There is a need for a radiation-free method and system for simulation of a radiographic image, such as an x-ray dental image. There is also a need for a simulated patient that has a range of motion similar to a live patient.

### SUMMARY

In accordance with the present invention, a system and a method are defined for simulating an x-ray image. The x-ray image may be a dental image, such as a dental radiograph, for example.

In one embodiment, the method may comprise obtaining, via a sensor associated with a simulated mouth, a signal emitted by an alignment device that is pointed at a location on or in the simulated mouth. The signal indicates the location. The method may further comprise determining a position and an angulation for the detected signal, correlating the position and the angulation to an image, and displaying the image on a template. In one example embodiment, the sensor may be a grid sensor that is applied over the simulated mouth. In another example embodiment, the sensor is an electromagnetic field device.

The method may further comprise obtaining a computerized tomography scan of a human mouth, including teeth on a mandible and maxilla within the human mouth, and mapping points on the computerized tomography scan of the human mouth to points on the simulated mouth.

Correlating the position and angulation to an image may further comprise mapping the position in the simulated mouth to a position on the computerized tomography scan and converting the computerized tomography scan to an x-ray projection at the angulation. The image may be cropped to a size consistent with a dental periapical or bitewing radiography and may be displayed on a template, such as a dental full mouth radiographic template.

In another example embodiment, the method may further comprise obtaining images comprising radiographs of a human mouth, including teeth on a mandible and maxilla within the human mouth and mapping the obtained images with locations in the simulated mouth. The radiographs may depict various positions within the human mouth and more than one angle for each position, and may be stored in storage of a computing device.

In one example embodiment, a determination that the angulation is not within a pre-determined range may be made, and in response to the determination, a message indicating an error may be displayed, as well as a message providing instructions to avoid making the error.

In another embodiment, a system for simulating an x-ray dental image, such as a radiograph, is provided. The system comprises an electromagnetic field device and a simulated head comprising a simulated mouth including a simulated mandible and maxilla and simulated teeth on the simulated mandible and maxilla, a first alignment device comprising a first sensor, a physical computing device comprising at least one processor and data storage comprising instructions executable by the at least one processor to cause the computing device to perform various operations. The operations may comprise detecting via the first sensor, a signal emitted by the alignment device that is pointed at a location on the simulated mouth, the signal indicating the location, determining a position and an angulation for the detected signal, correlating the position and the angulation to an image, and displaying the image on a radiographic template.

The first alignment device may comprise a sensor wire and a rod attached to a film holder. A second alignment device may be provided as well and may comprise a simulated x-ray cone comprising a first end, a second end, and a throughbore. The second sensor may be connected to the simulated x-ray cone at the first end and an alignment ring may be formed on the second end. A port may be connected to the ring for alignment to the rod, and the rod may be received in the port.

The system and method may be used for training to diagnose, provide a prognosis, monitor treatment, and guide treatment decisions for a disorder of the mouth.

These as well as other aspects and advantages of the synergy achieved by combining the various aspects of this technology, that while not previously disclosed, will become apparent to those of ordinary skill in the art by reading the following detailed description, with reference where appropriate to the accompanying drawings.

### BRIEF DESCRIPTION OF THE FIGURES

FIGS. 1a-c depict an exemplary instructional device in accordance with at least one embodiment;

FIG. 2 depicts an exemplary first alignment device in accordance with at least one embodiment;

FIGS. 3a-c depict an exemplary second alignment device in accordance with at least one embodiment;

FIG. 4 depicts an exemplary system comprising the first alignment device of FIG. 2 and second alignment device of FIGS. 3a-c coupled and positioned on the instructional device of FIGS. 1a-c, in accordance with at least one embodiment;

FIG. 5 depicts an exemplary computerized tomography scan taken from a human subject for use with the system of FIG. 4, in accordance with at least one embodiment;

FIG. 6a depicts an example full mouth template, in accordance with at least one embodiment;

FIG. 6b depicts an exemplary film placement for an oral radiograph in a mouth, in accordance with at least one embodiment; and

FIG. 7 depicts a simplified flow diagram of an example method that may be carried out simulate an x-ray image, in accordance with at least one embodiment.

### DETAILED DESCRIPTION

In the following detailed description, reference is made to the accompanying figures, which form a part thereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Radiographic technique training is needed to develop the radiographic skills for a number of health workers, such as dentists and dental students, dental assistants, and dental hygienists. However, current techniques rely on real dental radiology equipment. Such techniques are typically carried out in a dental radiology facility under supervision to ensure compliance with state and federal laws for radiation exposure, and thus training must be done when the facility is not in use by real patients. Furthermore, use of radiology equipment subjects a user to a risk of radiation exposure.

A system is provided to simulate a scan of part of the human body to obtain an x-ray image, such as a dental radiograph, allowing for radiographic technique training without use of radiation.

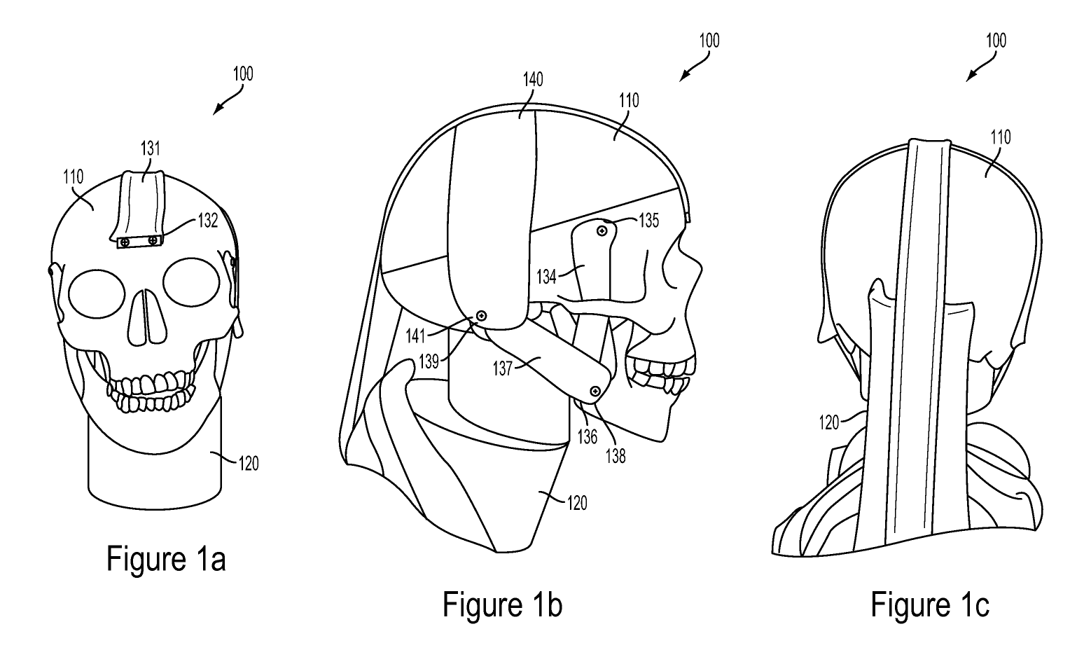

FIGS. 1a-c depict an exemplary instructional device 100 in accordance with at least one embodiment. The instructional device 100 may be used, among other things, to simulate an x-ray dental image, for example, a dental radiograph.

FIG. 1a depicts a frontal view of the instructional device 100, and FIGS. 1b and 1c depict side or lateral and posterior views thereof, respectively. The instructional device 100 may include a real human or simulated human head 110 mounted on a shaft 120 to maintain the head 110 in an upright position. The shaft may be mounted to a base 130.

The head 110 of the instructional device 100 includes a mouth comprising a maxilla and a mandible, and projections therefrom of simulated or real human teeth. Although a skull in skeletal form is shown as the instructional device 100, in some example embodiments the instructional device may comprise a head that resembles that of a live human, with real or simulated tissues and skin.

Whereas simulated human heads typically include a spring to open and close the mouth, the instructional device 100 includes elastic bands attached at various parts of the head 110. These elastic bands provide for movement of various parts of the head with respect to each other. Use of a spring to open and close a simulated mouth limits the range of motion for the mouth, thus rendering placement of a film in the mouth difficult. The elastic bands are positioned on the instructional device 100 to provide for a greater range of movement, simulating movement ranges a live human head is capable of.

A first elastic band 131 may be attached at a first end 132 to a location on a forehead of the head 110, as shown in FIG. 1a. The first elastic band 131 may extend across or span the head 110 in a direction toward the posterior of the head 110. A second end is attached to a location on one of the parietal or occipital bone locations or below the head 110. A second elastic band 134, shown in FIG. 1b, may attach at a first end 135 to a location on one of the sphenoid, zygomatic, or temporal bone locations of the head 110 and at a second end 136 to a location on the mandible of the head 110. A third elastic band 137 may attach at a first end 138 to a location on the mandible of the head 110 and at a second end 139 to a location on the occipital bone of the head 110. A fourth elastic band 140 may attach at a first end 141 to a location on the occipital bone and may extend across or span the head 110 to attach at a second end to a location on the occipital bone on the opposite side of the head 110. Fewer elastic bands may be present than those described. Additional elastic bands may be present to attach various other parts of the head to each other or to another part of the instructional device, such as the shaft 120 or the base 130.

The elastic bands may be attached to the head 110 via screws or other fasteners. In some example embodiments, the elastic bands may comprise a rubber or rubber-like material that can maintain stretch capacity over a prolonged period of time.

The elastic bands 131, 134, 137, and 140 allow for the maxilla and the mandible to move relative each other to provide a full range of motion, wherein full range of motion is defined as a range of motion simulating the range of motion a human mandible and maxilla. In operation, a user manually opens the simulated mouth of the head 110.

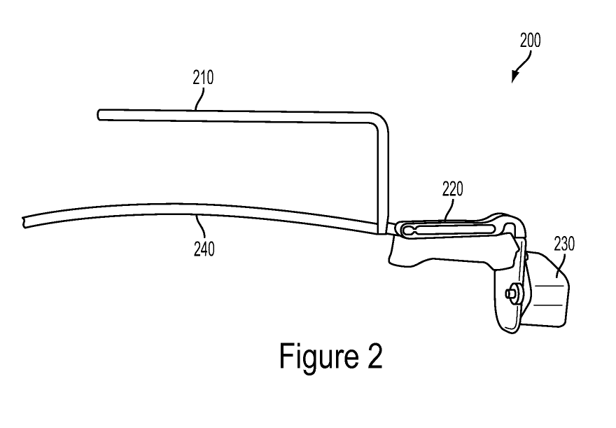

FIG. 2 depicts an exemplary first alignment device 200 in accordance with at least one embodiment. The first alignment device 200 may be used on an instructional device such as the instructional device 100 of FIGS. 1a-c. The first alignment device 200 comprises a rod 210, a film holder 220, and a first sensor 230. The first alignment device 200 may be a simulated radiographic alignment device.

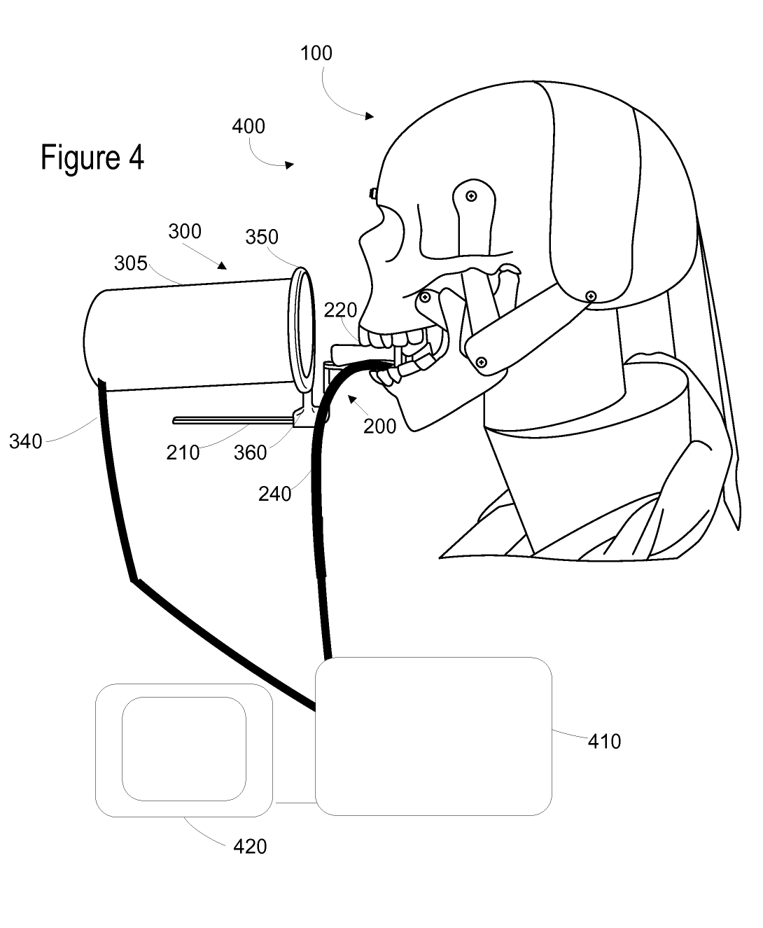

A sensor wire 240 may be attached to the first alignment device 200, as shown in FIG. 2. The sensor wire 240 may be an electromagnetic field (EMF) detector, in one example embodiment. The sensor wire 240 may be used to provide feedback from the first sensor 230 to a computing system, as will be described in further detail with respect to FIG. 4.

The rod 210 is an indicator rod attached at or near an end of the film holder 220 that may be manipulated or handled by a user to properly position the first sensor 230 with respect to a mouth on an instructional device, such as the instructional device 100. The rod may be made from a metal in some example embodiments. In one example embodiment, the rod may be aluminum. Other materials may be used for the rod 210 as well.

The film holder 220 may be a bite block for insertion into the mouth of the instructional device 100. The film holder 220 may be manually inserted within the mouth by a user. After insertion, the mouth is typically closed to obtain a simulated scan. The film holder 220 is sized and shaped to be held between the teeth on the maxilla and the teeth on the mandible when the mouth is closed. A film that would typically be used to obtain an x-ray image of an object is within the film holder.

The first sensor 230 may be a position sensor, and may be used to determine a position of the film inside the mouth of the instructional device 100. The first sensor 230 is attached to the film holder 220. Information obtained from the first sensor 230 is transmitted to a computing system for image production, as will be described in further detail below.

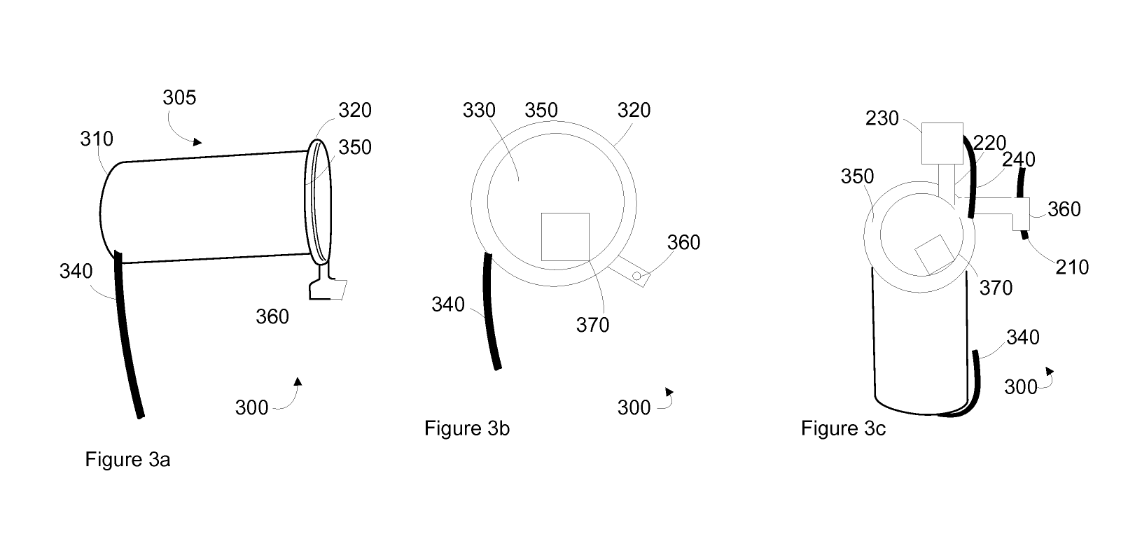

FIGS. 3a-c depict an exemplary second alignment device 300 in accordance with at least one embodiment. The second alignment device 300 may comprise a simulated x-ray cone.

FIG. 3a depicts a lateral view of the second alignment device 300. The second alignment device 300 comprises a cylindrical pipe 305 comprising a first end 310, a second end 320, and a throughbore 330 extending from the first end 310 to the second end 320. A sensor wire 340 may be attached to the first end 310 of the second alignment device 300. The sensor wire 340 may be an electromagnetic field (EMF) detector, in one example embodiment. Additionally, a ring 350 comprising an extension with a port 360 is attached to the second end 320.

The ring 350 may be an alignment ring, and the extension with the port 360 is configured to receive a rod, such as the rod 210, to align the first alignment device 200 to the second alignment device 300.

FIG. 3b depicts a superior view of the second alignment device 300. A second sensor 370 is present within the throughbore 330 of the x-ray cone 300 and serves to measure proximity of the ring to the first alignment device 200. The second sensor 370 may be affixed to an interior surface of the cylindrical pipe 305. The second sensor 370 is used to measure the proximity of the ring 350 to film holder 220 when they are coupled or in the process of being coupled.

A pre-determined distance between the first alignment device 200 and the second alignment device 300 may be set and stored in a computing system, such as the computing system 410 of FIG. 4. If the second sensor 370 detects that the position of the first sensor 230 falls outside of the pre-determined distance or range, or does not read the first sensor 230 within a certain distance (e.g., a few centimeters), the computing system will execute instructions to display an error message on a display screen, such as the display 420 of FIG. 4. If the sensor 370 detects that the position of the first sensor 230 of the first alignment device is located within the pre-determined distance or range, the second sensor 370 transmits such information to a computing device which may responsively execute instructions to display an image detected by the first sensor 230.

FIG. 3c depicts a superior oblique view of the second alignment device 300 of FIGS. 3a-b attached or coupled to the first alignment device 200. As shown in FIG. 3c, the rod 210 of the first alignment device 200 is positioned within the port 360 of the second alignment device 300.

FIG. 4 depicts an exemplary instructional system 400. The instructional system 400 comprises the first alignment device 200 of FIG. 2 coupled to the second alignment device 300 of FIGS. 3a-c, in position on the simulated human of FIGS. 1a-c. The first sensor wire 240 and the second sensor wire 340 are connected to a computing system 410 and a display 420.

The computing system 410 may be present within a base of the instructional device 100. Alternatively, the computing system 410 may be separate from and outside of the instructional device 100. The computing system 410 may include a processor, data storage, and logic. These elements may be coupled by a system or bus or other mechanism. The processor may include one or more general-purpose processors and/or dedicated processors, and may be configured to perform an analysis on the output from the sensor 340 and the sensor 240. An output interface may be configured to transmit output from the computing system to the display 420.

The computing system 410 may include instructions to convert a portion of a computerized tomography (CT) scan to an x-ray image in some example embodiments. In other example embodiments, the computing system 410 may be further configured to correlate position and angulation data with an image stored in data storage.

In operation, a user opens the mouth of the instructional device 100 and inserts the film holder 220 into the mouth at a desired position. Once the desired position is achieved, the user closes the mouth of the instructional device 100 so that the teeth bite down on the film holder. The user may also align the second alignment device with the first alignment device by sliding the extension with the port 360 over the rod 210. The second sensor 370 detects the position of the first alignment device and provides data regarding the position to the computing system via the second sensor wire 340. If the second sensor 370 detects that the first alignment device 200 is outside of a pre-determined distance or pre-determined acceptable range with respect to the second alignment device 300, the computing system 410 will execute instructions to display an error message on the display 420. If the second sensor 370 detects that the first alignment device 200 falls within the pre-determined distance or a pre-determined acceptable range, the computing system may execute instructions to display a message that the alignment is acceptable. Alternatively, the computing system may not display any message and the user may simply be allowed to move on to the next step in the training process.

After proper alignment of the first alignment device 200 with respect to the second alignment device 300 is attained, the user may further manipulate the film holder 220 by manual manipulation of the rod 210. When the desired position and angle for the film holder is achieved, the user may make a selection with the computing system indicating that an image is to be taken. When the computing system receives an indication of a selection, the computing system obtains a signal emitted by the first sensor wire 240 that indicates the location of a signal emitted by the first sensor 230 within the mouth. The indication may include data providing both a position within the mouth and an angle of the first sensor 230.

The computing system 410 correlates the position and angulation to an image. The computing system then executes instructions to display the image on a template, such as a dental full mouth radiographic template.

In one example embodiment, a CT scan of a human mouth, including teeth on a mandible and maxilla within the human mouth, is stored in the computing system 410. The CT scan may have points mapped and correlated to points on and within the mouth of the instructional device 100. When the computing system obtains the signal from the first sensor wire 240, the computing system may execute instructions to map the position and angulation data to a position on the CT scan and convert the CT scan to an x-ray projection image at the angulation. To perform the conversion, the computing system may use volume ray casting or another analogous technique to convert the image from grayscale to an x-ray projection. The x-ray projection image may be cropped to a size consistent with a dental periapical or bitewing radiograph.

In another example embodiment, images comprising radiographs of a human mouth, including teeth on a mandible and maxilla within the human mouth, may be obtained and stored in the computing system 410. The obtained images may be mapped to locations in the mouth of the instructional device 100, and may depict images taken at various angles for each location. A grid sensor may be embedded in the instruction device's gingiva, bone, and/or teeth to receive a signal emitted from the first sensor 230. When the computing system obtains the signal from the grid sensor, the computing system may execute instructions to correlate the position and angulation data to a stored image. The correlation may be performed using a global positioning technique (e.g., Global Positioning System (GPS)). The stored image that correlates indicates what an image would look like if taken by the film of the first alignment device within the mouth for the position and angle of the film holder.

In either embodiment, if the computing system makes a determination that the angulation or the position is not within a pre-set range, the computing system may execute instructions to display a message indicating an error via the display 420. Furthermore, the computing system may execute instructions displaying a message providing instructions for placement of one or both of the first and second alignment devices to avoid making the error.

A film is preferably angled with respect to both the maxillary teeth and the mandibular teeth. The film is preferably at an angle between 20° and 45° with respect to the maxillary teeth, for example. Films positioned at angles less than 20° and greater than 90° with respect to the maxillary and mandibular teeth are deemed to be clinically unacceptable; such data may be stored to provide a pre-set range of acceptable angulation for the film within a computing system, such as the computing system 410.

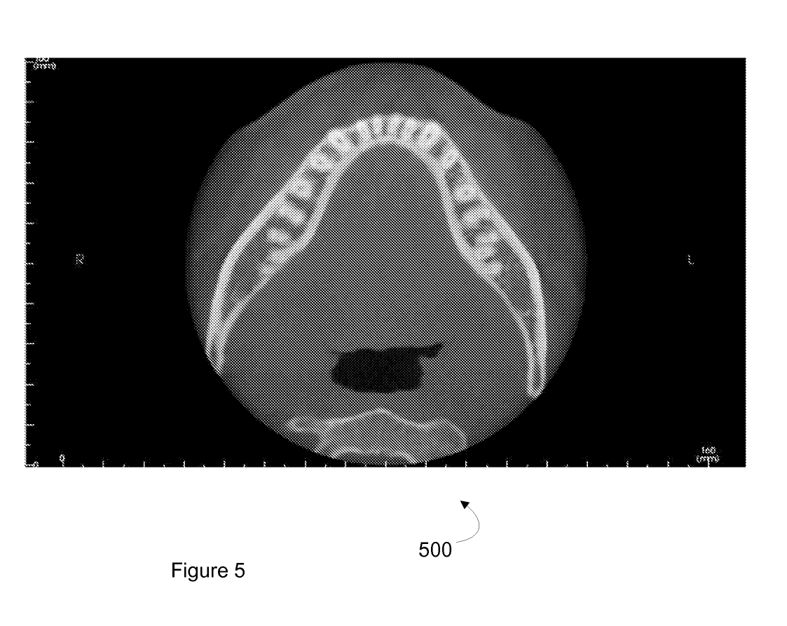

FIG. 5 depicts an exemplary CT scan 500 taken from a human subject for use with the system of FIG. 4, in accordance with at least one embodiment. The CT scan 500 may be obtained from a cone-beam computed tomography system (CBCT) or from a traditional computed tomography (CT) system. The CT scan 500 may include a three-dimensional reconstruction of human anatomy, namely, the teeth, oral and maxillofacial region (mouth, jaw, and neck), and ears, nose, and throat (ENT).

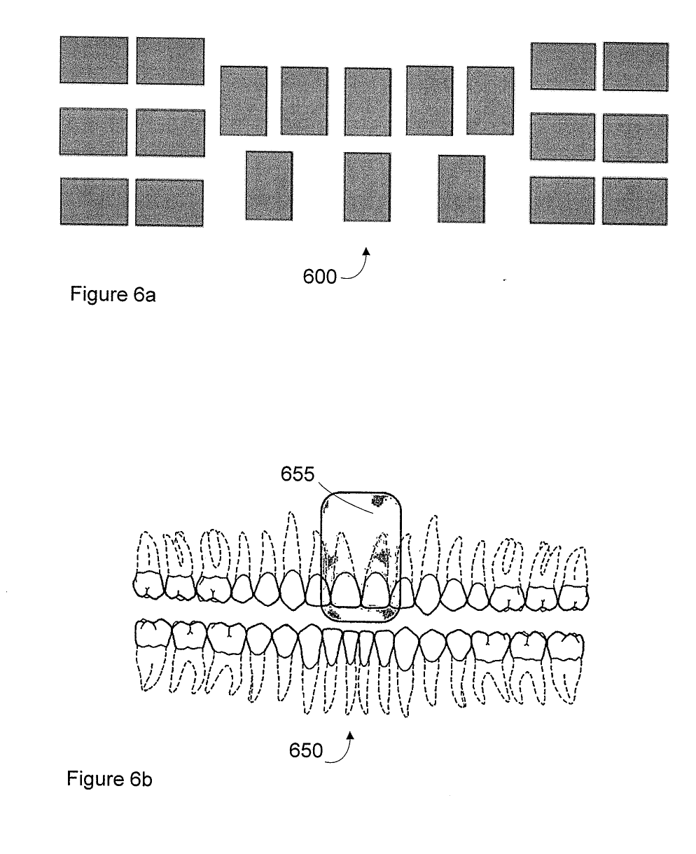

FIG. 6a depicts an example full mouth template 600, in accordance with at least one embodiment. The full mouth template 600 may be displayed on the display 420 of FIG. 4, and an obtained image may be positioned into one of the boxes in the template 600. The full mouth template 600 may be a template well-known in the industry, wherein various images taken from desired locations in the mouth are to be placed in boxes specific to such a position. The full mouth template 600 may comprise boxes indicating spots for eight posterior periapicals, four bitewings, and eight anterior periapicals.

FIG. 6b depicts a diagram of an exemplary film placement 650 for an oral radiograph in a mouth, in accordance with at least one embodiment. The box 655 indicates the location of the film over a section of the mouth.

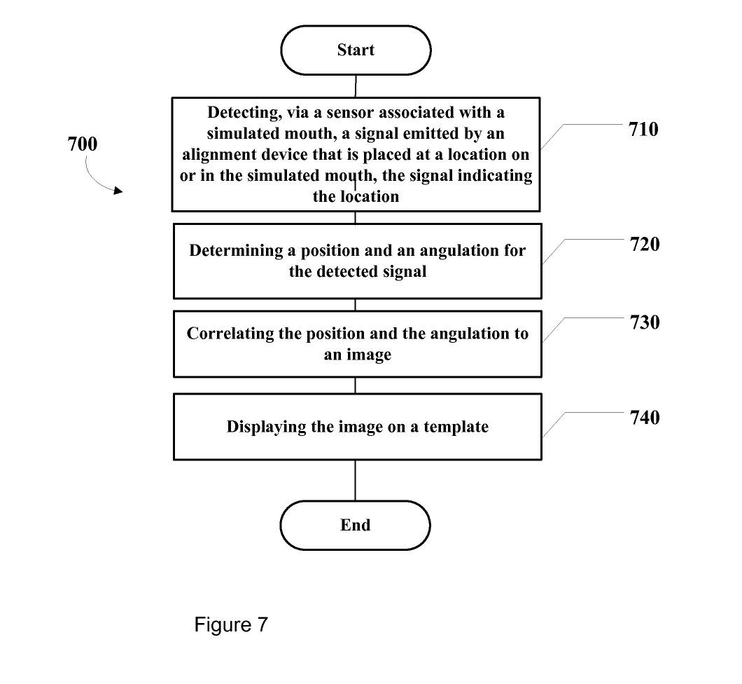

FIG. 7 depicts a simplified flow diagram of an example method 700 that may be carried out simulate an x-ray image, in accordance with at least one embodiment.

Method 700 shown in FIG. 7 presents an embodiment of a method that, for example, could be used with the instructional system 400. In addition, for the method 700 and other processes and methods disclosed herein, the flowchart shows functionality and operation of one possible implementation of the present embodiments. In this regard, each block may represent a module, a segment, or a portion of program code, which includes one or more instructions executable by a processor for implementing specific logical functions or steps in the process. The program code may be stored on any type of computer readable medium, for example, such as a storage device including a disk or hard drive. The computer readable medium may include a physical and/or non-transitory computer readable medium, for example, such as computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a computer readable storage medium, a tangible storage device, or other article of manufacture, for example. Alternatively, program code, instructions, and/or data structures may be transmitted via a communications network via a propagated signal on a propagation medium (e.g., electromagnetic wave(s), sound wave(s), etc.).

The method 700 allows for simulation of an x-ray dental image using a simulated human and alignment devices comprising sensors that communicate with a computing system. The simulated human may be the same or similar to the instructional device 100 of FIGS. 1a-c. The alignment devices may be the same or similar to the first alignment device 200 and the second alignment device 300 of FIGS. 2 and 3a-c. The method 700 may be used to train users in radiology techniques, such as how to perform one or more dental scans, for example.

Initially, the method 700 includes obtaining, via a sensor associated with a simulated mouth, a signal emitted by a sensor wire attached to an alignment device that is pointed at a location on or in the simulated mouth, at block 710. The signal indicates the location.

The method 700 then includes determining a position and an angulation for the detected signal, at block 720.

The method 700 includes correlating the position and the angulation to an image, at block 730.

The method 700 includes displaying the image on a template, at block 740. Prior to display on the template, the image may be cropped to a size consistent with a dental periapical or bitewing radiograph. The template may be a dental full mouth radiographic template, such as the template 600 of FIG. 6a.

The method 700 may additionally include obtaining a computerized tomography scan of a human mouth, including teeth on a mandible and maxilla within the human mouth, and mapping points on the computerized tomography scan of the human mouth, and correlating the mapped points to points on the simulated mouth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims, along with the full scope of equivalents to which such claims are entitled. It is also to be understood that the terminology used herein is for the purpose of describing particular embodiments only, and is not intended to be limiting.

1. A method for simulating an x-ray dental image comprising:
obtaining, via a sensor associated with a simulated mouth, a signal emitted by a sensor wire attached to an alignment device that is pointed at a location on or in the simulated mouth, the signal indicating the location;
determining a position and an angulation for the detected signal;
correlating the position and the angulation to an image; and
displaying the image on a template.

2. The method of claim 1, further comprising:
obtaining a computerized tomography scan of a human mouth, including teeth on a mandible and maxilla within the human mouth;
mapping points on the computerized tomography scan of the human mouth; and
correlating the mapped points to points on the simulated mouth.

3. The method of claim 2, the correlating the position and the angulation to an image further comprising:
mapping the position in the simulated mouth to a position on the computerized tomography scan;
converting the computerized tomography scan to an x-ray projection at the angulation; and
cropping the x-ray projection to a size consistent with a dental periapical or bitewing radiograph.

4. The method of claim 1, further comprising:
obtaining images comprising radiographs of a human mouth, including teeth on a mandible and maxilla within the human mouth; and
mapping the obtained images with locations in the simulated mouth;
wherein the radiographs depict various positions within the human mouth and more than one angle for each position.

5. The method of claim 1, further comprising:
storing the images in storage of a computing device.

6. The method of claim 1, wherein the sensor comprises a grid sensor embedded within gingiva of the simulated mouth.

7. The method of claim 1, wherein the simulated mouth has maxillary and mandibular articulation.

8. The method of claim 1, further comprising:
determining that the angulation is not within a pre-determined range, and in response to the determination, displaying a message indicating an error.

9. The method of claim 8, further comprising displaying a message providing instructions to avoid making the error.

10. The method of claim 1, wherein the sensor is an electromagnetic field device.

11. The method of claim 1, wherein the method is used to diagnose, prognose, or monitor treatment for a disorder of the teeth or gums.

12. The method of claim 1, wherein the method is used for radiographic technique training.

13. A system for simulating an x-ray dental image comprising:
a simulated head comprising:
a simulated mouth comprising a simulated mandible and a simulated maxilla and simulated teeth on the simulated mandible and the simulated maxilla;
an electromagnetic field detector;
a first alignment device comprising a first sensor;
a physical computing device comprising at least one processor and data storage comprising instructions executable by the at least one processor to cause the computing device to perform operations comprising:
detecting, via the first sensor, a signal emitted by the alignment device that is pointed at a location on the simulated mouth, the signal indicating the location;
determining a position and an angulation for the detected signal;
correlating the position and the angulation to an image; and
displaying the image on a radiographic template.

14. The system of claim 13, the first alignment device further comprising a rod attached to a film holder.

15. The system of claim 14, further comprising a second alignment device comprising:
a simulated x-ray cone comprising a first end, a second end, and a throughbore;
wherein a second sensor is connected to the simulated x-ray cone at the first end, an alignment ring is formed on the second end, and a port is connected to the ring for alignment to the rod.

16. The system of claim 15, wherein the computing device executes instructions to display an error message when the second alignment device is not positioned within a pre-determined distance from the first alignment device.

17. The system of claim 15, wherein the rod is received in the port to assemble the first alignment device to the second alignment device.

18. The system of claim 13, the instructions further comprising:
correlating points on a computerized tomography scan of a human mouth with points on the simulated mouth.

19. The system of claim 13, the instructions further comprising:
mapping the position in the simulated mouth to a position on the computerized tomography scan; and
converting the computerized tomography scan to an x-ray projection at the angulation.

20. The system of claim, further comprising:
images comprising radiographs of a human mouth, including teeth on a mandible and maxilla within the human mouth stored in the data storage.
